(19) United States
(12) Patent Application Publication
(10) Pub. No.: US 2006/0064121 A1
(43) Pub. Date: Mar. 23, 2006

(54) Apparatus for cutting an epithelial corneal flap

(75) Inventor: Antoine Sempe, Issy-Les-Moulineaux (FR)

Correspondence Address: YOUNG & THOMPSON, 745 SOUTH 23RD STREET, 2ND FLOOR, ARLINGTON, VA, 22202, US

(73) Assignee: MORIA S.A., ANTONY (FR)

(21) Appl. No.: 11/051,620

(22) Filed: Jan. 27, 2005

(30) Foreign Application Priority Data
Sep. 17, 2004 (FR) 04 09836

(51) Int. Cl. A61F 9/00

(52) U.S. Cl. 606/166

(57) ABSTRACT

Apparatus for cutting a corneal flap with: a fixing ring; a suction source; a cutter head including an oscillating blade; actuator elements for actuating the cutter head relative to the ring; control elements for controlling the settings for suction, oscillation, and actuation, of the blade so as to produce: a stage in where cutting of the corneal flap is started, the suction level is at a maximum, the cutter head advance speed is at a minimum, while the blade frequency oscillation is at a maximum; a stage of cutting the major portion of the corneal flap where the advance speed is maintained or increased while the oscillation speed and the suction level are maintained or decreased; and a third stage where the head speed is reversed, the oscillations being stopped and the suction level maintained at the level of the second stage.

Refractive surgery is performed on the cornea of the eye in order to modify the shape thereof towards correcting or improving its optical properties.

### BACKGROUND OF THE INVENTION

In this field, surgical procedures are constantly changing since account is being taken permanently and continuously of information, summaries, conclusions, and hypotheses that result from operations that have been performed and from their follow-up over several tens of years.

At present, some of the experimentation is directed towards performing an operation on the cornea which consists in removing an epithelial flap, taking action on the surface revealed thereby, and replacing the epithelial flap on the zone on which intervention has taken place, with this being done as well as possible given the quality of the epithelial flap.

One of the challenges of this procedure lies in preparing an epithelial flap of acceptable quality to enable it to be folded back down on the field of the intervention.

### OBJECT AND SUMMARY OF THE INVENTION

The object of the invention is to provide surgeons with means for performing such cutting of the epithelial flap under the best possible conditions.

To this end, the invention thus provides apparatus for cutting an epithelial corneal flap, the apparatus comprising:
a fixing ring that can be fixed onto the eye of the patient by suction;
suction source means connected to the ring and adjustable in terms of the level of suction that is created in the ring;
a cutter head mounted to move on the ring and guided relative thereto in a scanning plane, said cutter head comprising:
a blade with an active edge that is blunt and rectilinear and guide members for guiding the blade in displacement relative to the cutter head and parallel to said active edge;
driver means for driving the blade in reciprocating motion at adjustable frequency relative to said cutter head; and
actuator means for moving the cutter head at adjustable speed relative to the ring, the apparatus further comprising control means for controlling the above-mentioned suction, actuator, and driver means so as to create a cutting cycle comprising:
a first stage of starting cutting the corneal flap in which the suction level is at a maximum and in which the speed of advance of the cutter head is established before cutting starts at a minimum value while the frequency of oscillation of the blade is at a level of maximum value;
a second stage of cutting the major portion of the corneal flap in which the speed of advance is increased while the frequency of oscillation and the level of suction are decreased, or at least conserved; and
a third stage during which the speed of the head is reversed, the oscillations being stopped and the level of suction being maintained at the decreased level of the second stage, or being decreased further.

Experiments have shown that using a blunt cutter blade makes it possible to dissociate the cornea in lamellar manner via its cleavage planes, i.e. planes representing a change in mechanical characteristics between corneal layers. Thus, for example, the epithelial layer rests on a membrane known as Bowman's membrane which presents greater resistance to penetration than does the epithelial layer itself. Thus, by using a blunt blade, it is possible to perform lamellar dissection down to the top of Bowman's membrane. The term “blunt” blade should be understood as a blade whose cutting edge is not very sharp because it has been sharpened in a particular manner and which might include a special surface finish. Nevertheless, there remains the problem of how to start cutting. In prior techniques, cutting has been started by means of a sharp blade like a trephine that is caused to penetrate into the cornea down to a depth that is substantially equal to the assumed thickness of the epithelial layer, after which the blade is changed so as to proceed with lamellar dissection by means of a blunt blade. That procedure is complicated and leads to uncertainties causing the surgical act to lack uniformity and repeatability. By adjusting parameters such as suction level, speed of advance, and frequency of oscillation of the blade, it has been found by experiment that the above-specified sequence of stages enables cutting to be started in satisfactory manner, after which it is possible to proceed with cutting of the lamellar dissection type, likewise in satisfactory manner, while also acting under conditions that make it possible to eliminate practically all risk of the blade perforating Bowmanis membrane. Finally, the third stage is defined so that return of the cutter blade takes place while minimizing potential occasions for trauma to the epithelial flap that has just been made.

Preferably, the above-mentioned control means comprise a pedal coupled to the above-mentioned suction, drive, and actuator means via programmable computer means. It is also advantageous for the time during which each stage takes place to be programmed, while the start of each stage is triggered by the user. All of the parameters could be fully programmed by the user or could be fully predetermined on manufacture.

### BRIEF DESCRIPTION OF THE DRAWING

Other characteristics and advantages of the invention appear from the following description of an embodiment.

Reference is made to the accompanying drawing, in which:

FIG. 1 is a diagrammatic overall view of apparatus in accordance with the invention; and

FIG. 2 is a graph showing the three operating stages I, II, and III of the apparatus in accordance with the invention.

### MORE DETAILED DESCRIPTION

The figures are diagrams showing the apparatus of the invention. In a manner that is known and similar to the microkeratomas used for the refractive surgery known as “laser-assisted in-situ keratomileusis” (lasik), the apparatus comprises a fixing ring 1 for applying against the eyeball 2 and held thereagainst by suction established in an annular chamber 3 that is closed by the eyeball 2, using a vacuum pump 4 having conventional means 12 for adjusting suction level and thus the vacuum that is established in the annular chamber 3.

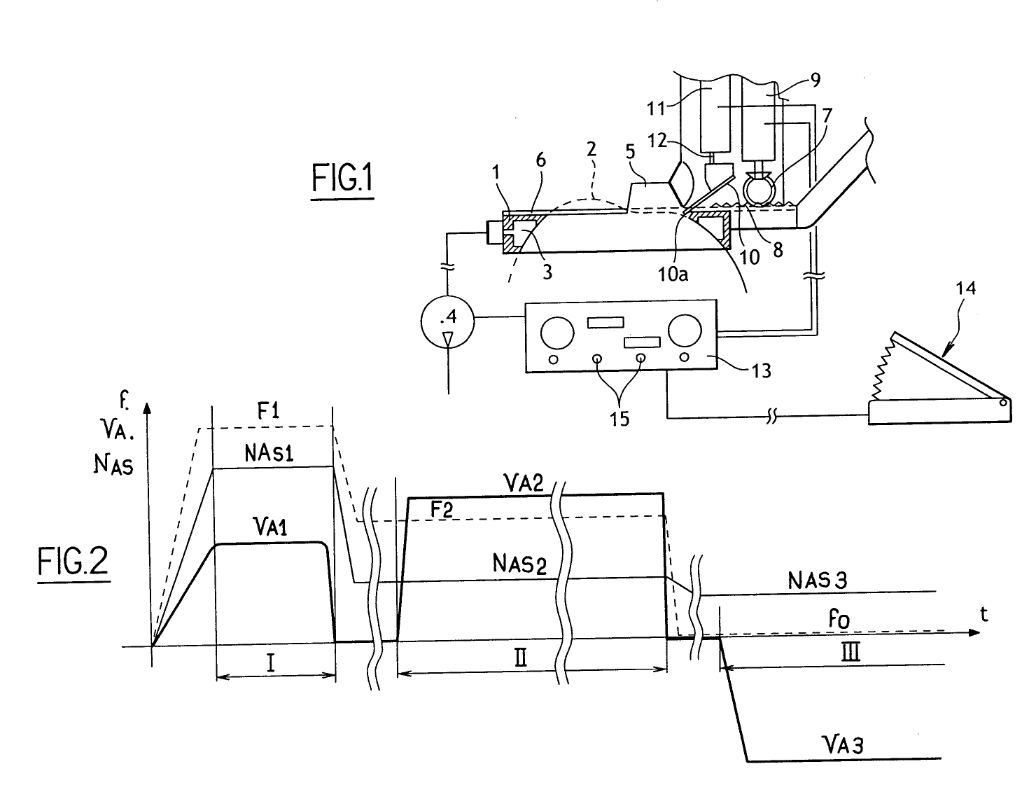

The ring defines a scanning plane for a cutter head 5 that is to move in said plane following a path that is rectilinear or circular under guidance from guide means co-operating with the ring and the cutter head. In FIG. 1, the guide means are constituted by rectilinear slideways 6 and the means for driving the head 5 in the slideways 6 are represented diagrammatically by co-operation between a pinion and a stationary rack 8. The pinion 7 is driven by a motor 9 of adjustable speed. Naturally, numerous other mechanisms can be implemented to provide these displacements.

Inside the cutter head, the apparatus has a cutter blade 10 mounted to slide in a housing that extends substantially transversely to the path followed by the cutter head 5, the cutter blade having an active edge 10a that is blunt. It is driven with reciprocating motion parallel to its active edge by means of a motor 11 and an eccentric 12. The frequency of oscillation depends on the speed of rotation of the eccentric 12 which is adjustable by means for controlling the motor 11.

The apparatus includes an electronic console 13 for controlling the keratome and suitable for displaying operating setpoints for each of the motor members such as the pump 4, the motor 9, and the motor 11, with this electronic control unit receiving as inputs signals that come from the surgeon such as, for example: signals coming from a pedal 14 operated by the foot of the surgeon. The electronic unit 13 also has means 15 for displaying the above-mentioned setpoint values so as to determine an operating cycle that is specific to the patient being operated and as has been determined by the surgeon who has examined the patient. The surgeon may have a plurality of available control pedals, all connected to the electronic unit. For example one pedal can serve to trigger suction, another pedal (having a plurality of thresholds) can serve to control cutting, and a third pedal can serve to return the cutter head.

At the time of the operation, the surgeon determines the various parameters determining the various setpoints for operation of the apparatus. It is in this way that a first frequency of oscillation F1 for the blade 10 is selected together with a first suction level NAS1 to be established in the ring, and a first speed of advance VA1 for the head along its path. A first pulse or first depression of the pedal 14 (or using a separate pedal) triggers operation of stage I as shown in FIG. 2. The cutter head is at the beginning of its path and not making any contact with the cornea, so there is time for the reference values to be reached before any action begins to be taken on the eye. Cutting is thus started in application of the variables F1, NAS1, and VA1, with this taking place for a length of time that can either be predetermined, or else left under the control of the surgeon who can release the pedal when cutting is deemed to have been started. At this instant, the speed of advance of the head drops to zero, while the frequency of oscillation of the blade reduces as to come down to a reference value F2, and likewise a lower suction level NAS2 is established in the ring 3. It is possible for F2 and NAS2 to be maintained equal to F1 and NAS1, but under no circumstances should they be any greater.

The surgeon then issues a second pulse or depresses the pedal 14 a second time, and cutting of the epithelial flap continues with the values F2, NAS2, and a speed of advance VA2 that is greater than the preceding speed VA1. In the same manner, this operation of cutting the main portion of the corneal flap is stopped, either in preprogrammed manner or on the initiative of the surgeon. At the end of this second stage referenced II in FIG. 2, the frequency of oscillation drops to zero as does the speed of advance, while the suction level is maintained at its threshold NAS2 or is reduced further to the level NAS3.

A third pulse on the pedal 14 or a control action on a separate pedal causes the travel direction of the cutter head 5 to reverse relative to the ring 1 and Lo travel at a reference speed VA3 so as to be able to withdraw the cutter head after making the epithelial flap and putting into place the laser bombardment for modifying the surface revealed by forming this lamellar flap. The surgeon terminates the surgery by folding the flap back down on the field that has just been sculpted by means of the laser.

It should be observed that the above-mentioned programming of the various parameters of the procedure can include steps of comparing physical magnitudes such as the magnitudes of the continuously-sensed power supply currents to the motors, with predetermined values for said magnitudes and representative of performing the respective stages of the surgical procedure.

What is claimed is:

1. Apparatus for cutting an epithelial corneal flap, the apparatus comprising:
a fixing ring that can be fixed on the eye of the patient by suction;
suction source means connected to the ring with the level of the suction created in the ring being adjustable;
a cutter head mounted to move on the ring and guided relative thereto in a scanning plane, said cutter head comprising a blade with an active edge that is blunt and rectilinear, guide members for guiding travel of the blade relative to the cutter head parallel to the above-mentioned active edge, and driver means for driving the blade in reciprocating motion of adjustable frequency relative to the cutter head;
actuator means for actuating movement of the cutter head relative to the ring at an adjustable speed VA, the apparatus including means for controlling said suction means, said driver means, and said actuator means so as to create a cutting cycle comprising:
a first stage in which cutting of the corneal flap is started and in which the suction level is at a maximum and in which the speed of advance of the cutter head is established before cutting starts at a minimum value, while the frequency of oscillation of the blade is established at a value of maximum level;
a second stage of cutting the greater part of the corneal flap during which the speed of advance is maintained or increased while the frequency of oscillation and the level of suction are decreased or at least retained; and
a third stage in which the speed of the head is reversed, the oscillations being stopped and the suction level being maintained at the lower level of the second stage.

2. Apparatus according to claim 1, wherein the above-mentioned control means comprise a pedal coupled to the above-mentioned suction, drive, and actuator means via programmable computer means.

3. Apparatus according to claim 1, including means for programming the time during which each stage takes place while the start of each stage is triggered by the user.
